(19) United States
(12) Patent Application Publication
(10) Pub. No.: US 2020/0297549 A1
(43) Pub. Date: Sep. 24, 2020

(54) FLEXIBLE HOLLOW STRUCTURE, ADHESIVE TAPE, AND FUNCTIONAL ADHESIVE SHEET

(71) Applicants: Toshihiro KANEMATSU, Kanagawa (JP); Koji IWASAKI, Kanagawa (JP)

(72) Inventors: Toshihiro KANEMATSU, Kanagawa (JP); Koji IWASAKI, Kanagawa (JP)

(21) Appl. No.: 16/820,232

(22) Filed: Mar. 16, 2020

(30) Foreign Application Priority Data
Mar. 20, 2019 (JP) 2019-053399

(51) Int. Cl. A61F 13/45; A61F 13/505

(52) U.S. Cl. CPC A61F 13/45; A61F 2013/5147; A61F 13/505

(57) ABSTRACT

Provided is a flexible hollow structure including a plurality of hollow cell sections each having an opening at one end, wherein when the area of a cross-section of each hollow cell section positioned closely to the opening and approximately in parallel with the opening is X (mm²), an opening area of the opening is 0.2X or greater but 0.7X or less.

### CROSS-REFERENCE TO RELATED APPLICATIONS

The present application claims priority under 35 U.S.C. § 119 to Japanese Patent Application No. 2019-053399 filed Mar. 20, 2019. The contents of which are incorporated herein by reference in their entirety.

### BACKGROUND OF THE INVENTION

### Field of the Invention

The present disclosure relates to a flexible hollow structure, an adhesive tape, and a functional adhesive sheet.

### Description of the Related Art

Percutaneous absorption preparations (hereinafter, may also be referred to as “patches”) need to efficiently express medical efficacy (drug release and drug cutaneous absorption) and need also to have close adhesiveness with skin and low skin stimulation.

Some skin stimuli are attributable to external forces. It is important to reduce skin stimulation due to such external forces. Particularly, an issue involved in peeling patches from skin is to reduce exfoliation of stratum corneum of the skin by the adhesive force of the patches.

As the method for reducing exfoliation of stratum corneum of the skin, for example, there has been a known method of varying the adhesive force in the peeling direction. There is a tendency that the greater the peeling angle, the lower the adhesive force. However, because skin has stretchability, even if an attempt to peel a patch by a large peeling angle is made, the skin deforms by following the patch in the peeling direction, failing to make the peeling angle large. Meanwhile, it has been known that the adhesive force is also dependent on the peeling speed, and making the peeling speed lower makes the adhesive force lower.

It is possible to obtain the effect of making the adhesive force low by using a soft plaster (adhesive+drug) such as gel, and making the thickness of a plaster large. However, if the plaster is too soft, there is a risk that a cohesive failure (breakage in the plaster section) occurs during peeling. If the thickness of the plaster is increased, the concentration of the drug (the ratio of the drug to the adhesive) is reduced. This leads to a problem that the drug release efficiency lowers and medical efficacy properties are degraded.

Hence, it has been proposed that a deformable hollow structure and an adhesive be used in a tape agent in order to provide an excellent close adhesiveness during attachment and to reduce exfoliation of stratum corneum during peeling (for example, see Japanese Unexamined Patent Application Publication No. 2018-145128).

### SUMMARY OF THE INVENTION

According to one aspect of the present disclosure, a flexible hollow structure of the present disclosure includes a plurality of hollow cell sections each having an opening at one end. When the area of a cross-section of each hollow cell section positioned closely to the opening and approximately in parallel with the opening is X (mm²), an opening area of the opening is 0.2X or greater but 0.7X or less.

### BRIEF DESCRIPTION OF THE DRAWINGS

FIG. 1A is a view illustrating an example of a hollow cell section of a flexible hollow structure of the present disclosure;

FIG. 1B is a view illustrating another example of a hollow cell section of a flexible hollow structure of the present disclosure;

FIG. 1C is a view illustrating another example of a hollow cell section of a flexible hollow structure of the present disclosure;

FIG. 1D is a view illustrating another example of a hollow cell section of a flexible hollow structure of the present disclosure;

FIG. 2A is a view illustrating an example shape of an opening of a hollow cell section of an existing flexible hollow structure;

FIG. 2B is a view illustrating an example shape of an opening of a hollow cell section of a flexible hollow structure of the present disclosure;

FIG. 2C is a view illustrating another example shape of an opening of a hollow cell section of a flexible hollow structure of the present disclosure;

FIG. 2D is a view illustrating another example shape of an opening of a hollow cell section of a flexible hollow structure of the present disclosure;

FIG. 2E is a view illustrating another example shape of an opening of a hollow cell section of a flexible hollow structure of the present disclosure;

FIG. 2F is a view illustrating another example shape of an opening of a hollow cell section of a flexible hollow structure of the present disclosure;

FIG. 3A is a view illustrating an example of a flexible hollow structure of the present disclosure;

FIG. 3B is a view illustrating another example of a flexible hollow structure of the present disclosure;

FIG. 3C is a view illustrating another example of a flexible hollow structure of the present disclosure;

FIG. 3D is a view illustrating another example of a flexible hollow structure of the present disclosure;

FIG. 4A is a view illustrating an example of an adhesive sheet of the present disclosure;

FIG. 4B is a view illustrating another example of an adhesive sheet of the present disclosure;

FIG. 4C is a view illustrating another example of an adhesive sheet of the present disclosure;

FIG. 4D is a view illustrating another example of an adhesive sheet of the present disclosure;

FIG. 4E is a view illustrating another example of an adhesive sheet of the present disclosure;

FIG. 4F is a view illustrating another example of an adhesive sheet of the present disclosure;

FIG. 4G is a view illustrating another example of an adhesive sheet of the present disclosure;

FIG. 4H is a view illustrating another example of an adhesive sheet of the present disclosure;

FIG. 4I is a view illustrating another example of an adhesive sheet of the present disclosure;

FIG. 4J is a view illustrating another example of an adhesive sheet of the present disclosure;

FIG. 4K is a view illustrating another example of an adhesive sheet of the present disclosure;

FIG. 4L is a view illustrating another example of an adhesive sheet of the present disclosure;

FIG. 5A is a view illustrating an example of a functional adhesive sheet of the present disclosure;

FIG. 5B is a view illustrating another example of a functional adhesive sheet of the present disclosure;

FIG. 5C is a view illustrating another example of a functional adhesive sheet of the present disclosure;

FIG. 5D is a view illustrating another example of a functional adhesive sheet of the present disclosure;

FIG. 5E is a view illustrating another example of a functional adhesive sheet of the present disclosure;

FIG. 5F is a view illustrating another example of a functional adhesive sheet of the present disclosure;

FIG. 6A is a view illustrating another example of a functional adhesive sheet of the present disclosure;

FIG. 6B is a view illustrating another example of a functional adhesive sheet of the present disclosure; and

FIG. 7 is a view illustrating an example of an adhesive sheet of Example.

### DESCRIPTION OF THE EMBODIMENTS

### (Flexible Hollow Structure)

A flexible hollow structure of the present disclosure includes a plurality of hollow cell sections each having an opening at one end. When the area of a cross-section of each hollow cell section positioned closely to the opening and approximately in parallel with the opening is X (mm²), an opening area of the opening is 0.2X or greater but 0.7X or less. The flexible hollow structure further includes other members as needed.

The present inventors have obtained the following finding as a result of studying a flexible hollow structure applicable to, for example, an article that is excellent in close adhesiveness and water permeability during attachment, and that can prevent an adhesive from remaining on skin and stratum corneum from being exfoliated when the article is peeled from skin.

According to existing techniques, a tape agent aiming for having excellent close adhesiveness during attachment and for being able to reduce exfoliation of stratum corneum during peeling uses a hollow structure including a plurality of hollow sections separated by partitioning walls and each having an opening in at least one surface. However, because the adhesive and the hollow structure only contact each other at the ends of the partitioning walls at the opening side, the hollow structure has a problem that the adhesive force between the adhesive and the adherend is greater than the adhesive force between the adhesive and the hollow structure to have the adhesive wholly or partially broken by stress when the tape agent is peeled from, for example, skin, to have the adhesive remain on skin.

It has been found that with an opening area of an opening of each hollow cell section, which has the opening at one end, set to 0.2X or greater but 0.7X or less with respect to an area X of a cross-section of the hollow cell section positioned closely to the opening and approximately in parallel with the opening, the flexible hollow structure of the present disclosure can have sufficient areas of contact between the adhesive to be provided on the opening regions and the hollow structure. Moreover, with the opening area of the opening of each hollow sell section set to 0.2X or greater but 0.7X or less with respect to the area X, the flexible hollow structure has been found applicable to, for example, an article that is excellent in close adhesiveness and water permeability during attachment, and that can prevent the adhesive from remaining on skin and stratum corneum from being exfoliated when the article is peeled from skin.

The present disclosure has an object to provide a flexible hollow structure applicable to, for example, an article that is excellent in close adhesiveness and water permeability during attachment, and that can prevent an adhesive from remaining on skin and stratum corneum from being exfoliated when the article is peeled from skin.

The present disclosure can provide a flexible hollow structure applicable to, for example, an article that is excellent in close adhesiveness and water permeability during attachment, and that can prevent an adhesive from remaining on skin and stratum corneum from being exfoliated when the article is peeled from skin.

### <Hollow Cell Section>

The hollow cell section is a structure including a hollow space defined by a partitioning wall, and has an opening at one end.

The flexible hollow structure of the present disclosure includes a plurality of hollow cell sections. When the area of a cross-section of each hollow cell section positioned closely to the opening and approximately in parallel with the opening is X (mm²), an opening area of the opening is 0.2X or greater but 0.7X or less.

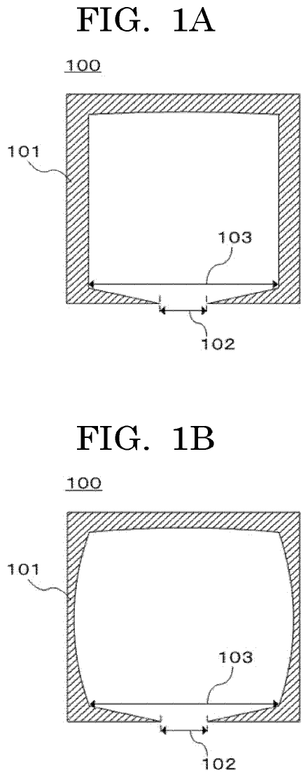

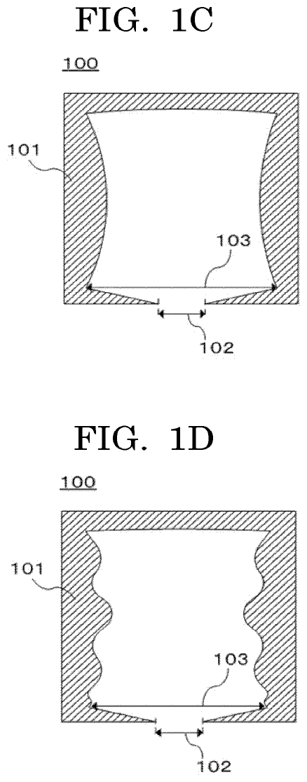

“Close to the opening of the hollow cell section” means an immediately underlying position with respect to the opening of the hollow cell section toward the hollow cell section. That is, “close to the opening of the hollow cell section” means a position shallowly inward from the opening into the hollow cell section (inside the hollow cell section). That is, “close to the opening of the hollow cell section” means the position denoted by 103 in FIG. 1A to FIG. 1D.

“The cross-section of the hollow cell section positioned closely to the opening and approximately in parallel with the opening” means a cross-section that is positioned at an immediately underlying position with respect to the opening of the hollow cell section toward the hollow cell section and is approximately in parallel with the surface, in which the opening is formed.

In the flexible hollow structure of the present disclosure, what is meant by that “when the area of the cross-section of the hollow cell section positioned closely to the opening and approximately in parallel with the opening is X (mm²), an opening area of the opening is 0.2X or greater but 0.7X or less” is that when the area of “the cross-section of the hollow cell section that is positioned closely to the opening and approximately in parallel with the opening, and that is observable when the opening is seen in the plan-view perspective from a position to look into the hollow cell section” is X (mm²), the opening area of the opening, i.e., the area of the actually open region is 0.2X or greater but 0.7X or less.

“The cross-section of the hollow cell section positioned closely to the opening and approximately in parallel with the opening” will be described in detail with reference to the drawings.

FIG. 1A to FIG. 1D are views illustrating examples of the hollow cell section of the flexible hollow structure of the present disclosure. The hollow cell section 100 has a partitioning wall 101 and an opening 102. There are not only a case where the cross sections of the hollow cell section positioned approximately in parallel with the opening have an approximately constant area from the bottom surface side to the opening side as illustrated in FIG. 1, but also a case where the cross-sectional shapes (the cross-sections positioned approximately in parallel with the opening) sectioned orthogonally to the direction extending from the bottom surface side to the opening side of the hollow cell section have variable areas depending on the position at which a cross-section is taken when the partitioning wall 101 of the hollow cell section 100 does not have a constant thickness as illustrated in FIG. 1B to FIG. 1D. Therefore, in the present disclosure, comparison is made between the opening area of the opening 102 and the area X of the cross-section 103 positioned closely to the opening (i.e., shallowly inward into the hollow cell section from the opening) and approximately in parallel with the opening, except the partitioning wall, in which the opening is formed.

The shape of the cross-section of the hollow cell section positioned approximately in parallel with the opening of the hollow cell section is not particularly limited and may be appropriately selected depending on the intended purpose. Examples of the shape of the cross-section include honeycomb (hexagonal) shapes, quadrangular shapes other than hexagonal shapes, polygonal shapes such as pentagonal shapes, and circular shapes. Among these shapes, honeycomb (hexagonal) shapes are preferable. When the shape of the cross-section of the hollow cell section positioned approximately in parallel with the opening is a honeycomb (hexagonal) shape, a honeycomb structure, which is the hollow structure, deforms in response to a press force during attachment. This facilitates stress relaxation, to make the distribution of the adhesive force of the adhesive uniform. When, for example, there is an external impact (e.g., rubbing by clothes) during use, the honeycomb structure bends and absorbs the impact, to distribute and decay the impact force that may reach the adhesive region, to reduce stimulation on the skin. During peeling, the honeycomb structure deforms in response to the peeling force and makes the peeling angle large, allowing expectation for reduction in the adhesive force. Moreover, because the adhesive is bound only on part of the partitioning walls of the hollow sections of the honeycomb structure, the structure can easily deform, allowing expectation for reduction in the adhesive force. These effects serve to reduce the adhesive force on the skin and consequently suppress exfoliation of keratinocytes.

It is preferable that the area of the cross-section of the hollow cell section positioned approximately in parallel with the opening be approximately constant. What is meant by that “the area of the cross-section of the hollow cell section positioned approximately in parallel with the opening is approximately constant” is that the cross-sections of the hollow cell section in the direction orthogonal to the direction from the bottom surface side to the opening side of the hollow cell section have a constant area. It is preferable that such a hollow cell section have a partitioning wall having an approximately constant thickness. When the partitioning wall of the hollow cell section has an approximately constant thickness, the strength of the flexible hollow structure can be optimized.

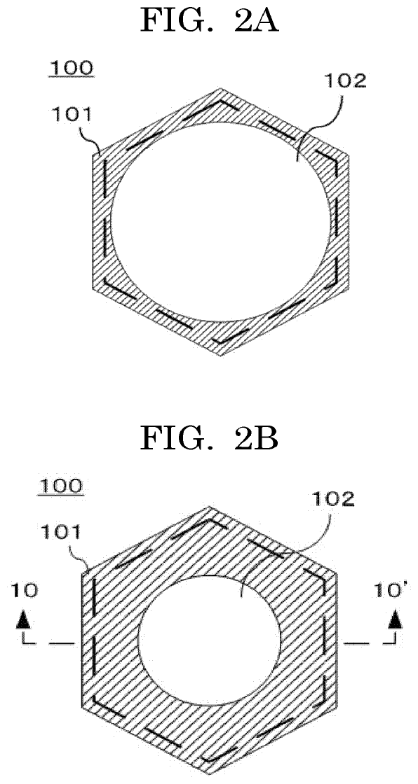

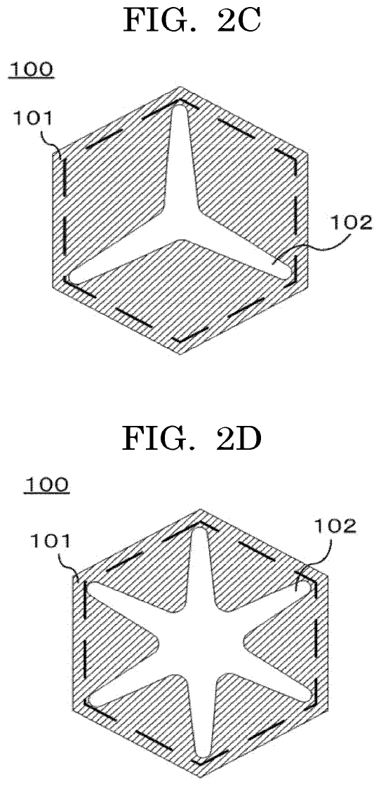

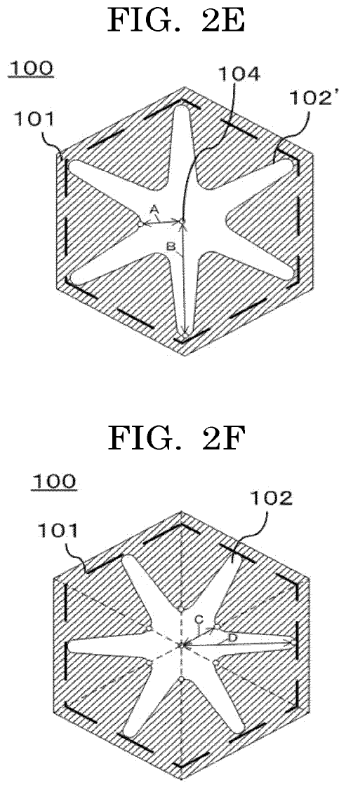

Next, the size of the area that may be possible as the opening area of the opening with respect to the area X of the cross-section described above will be described with reference to FIG. 2A to FIG. 2F. The clotted line represents the boundary between the interior of the hollow cell section and the partitioning wall positioned as the circumferential side surface of the hollow cell section.

FIG. 2A illustrates an example of a plan view of a hollow structure of an existing tape agent at the opening side. Here, description will be made about an example where the shape of the hollow cell section seen in the plan-view perspective from the opening side is a hexagonal shape. The present disclosure is not limited to this example.

As illustrated in FIG. 2A, in the hollow structure 100 used in the existing technique, the opening 102 is almost inscribed with the partitioning wall 101, and the area over which the adhesive can be applied is small.

FIG. 2B to FIG. 2F illustrate example plan views of the flexible hollow structure of the present disclosure at the opening side of the hollow cell section. FIG. 2B to FIG. 2F are examples in which the opening area is adjusted in a manner that the opening area of the opening 102 is 0.2X or greater but 0.7X or less. As illustrated in FIG. 2B to FIG. 2F, with the opening area adjusted to 0.2X or greater but 0.7X or less, the area over which the adhesive can be applied can be secured more largely than in the case of the opening illustrated in FIG. 2A. The cross section 10-10′ illustrated in FIG. 2B is a cross section taken from FIG. 1A.

The opening area is 0.2X or greater but 0.7X or less, preferably 0.2X or greater but 0.6X or less, and more preferably 0.2X or greater but 0.5X or less. When the opening area is 0.2X or greater but 0.7X or less, the area over which the adhesive can be applied can be secured largely, making it possible to prevent the adhesive from being broken by failing to endure stress during peeling from, for example, skin. Moreover, because the area over which the adhesive can be applied can be secured largely, the adhesive region is wide, making it possible to improve close adhesiveness. Further, because the area over which the adhesive can be applied can be secured largely, the adhesive can be suppressed in the thickness, making the adhesive region more deformable and have an improved followability to the surface of, for example, skin, making it possible to improve close adhesiveness. Moreover, with a small adhesive thickness, moisture can easily permeate the hollow structure, making it possible to reduce stimulation on skin due to, for example, damp with sweat.

The shape of the opening of the hollow cell section is not particularly limited and may be appropriately selected depending on the intended purpose so long as the opening satisfies the condition that the opening area is 0.2X or greater but 0.7X or less. Examples of the shape of the opening include the shape satisfying the conditions described below.

As the shape of the opening of the hollow cell section, it is preferable that at least two points on the contour of the opening of the hollow cell section be positioned at different distances from the center of the opening.

“At least two points on the contour of the opening of the hollow cell section being positioned at different distances from the center of the opening” will be described with reference to the drawings.

FIG. 2E is an example illustrating a case where the shape of the opening of the hollow cell section is approximately a star shape. In FIG. 2E, the contour 102′ of the opening has a shape represented by an aggregation of dots positioned at different distances from the center in the bottom surface of the hollow cell section 100 at the opening side. Here, “the center of the opening” means the center of the shape constituting the opening. “The center of the opening” may be “the center of gravity of the opening”.

In the shape of the opening illustrated in FIG. 2E, there exists on the same contour, a point A that is at a distance from the center 104 of the hollow cell section 100 and a point B that is at a different distance from the center 104 of the hollow cell section 100. In this way, with at least two points on the contour of the opening of the hollow cell section positioned at different distances from the center of the opening, a range of motion in the bottom surface at the opening side is large, to make the adhesive region easily deformable, making it possible to improve followability to the surface of, for example, skin, and improve close adhesiveness.

As the shape of the opening of the hollow cell section, it is preferable that the contour of the opening of the hollow cell section have an inflection point, and that a contour portion at which the inflection point is present be a curve.

“The inflection point” refers to a point at which a curve on a plane changes curving directions. For example, it is preferable that the inflection point be a curve as represented by the points A and B in FIG. 2E. When the inflection point is a curve, stress concentration can be prevented when the adhesive applied is peeled, making the adhesive less likely to remain on, for example, skin.

Further, the shape of the opening of the hollow cell section may be a shape that, when the opening of the hollow cell section is seen in a plan-view perspective, has a contour having six points positioned on straight lines coupling the vertices of the honeycomb shape to the center of the opening and a point positioned at a greater distance from the center of the opening than at least one of the six points.

Here, “the shape of the opening having a contour having six points positioned on straight lines coupling the vertices of the honeycomb shape to the center of the opening and a point positioned at a greater distance from the center of the opening than at least one of the six points” will be described with reference to the drawings.

FIG. 2F is another example illustrating a case where the shape of the opening of the hollow cell section is approximately a star shape. FIG. 2F illustrates an example in which the shape of the opening illustrated in FIG. 2E is rotated by 30 degrees. First, “the six points on the contour of the opening positioned on straight lines coupling the vertices of the honeycomb shape to the center of the opening” refer to, for example, the point denoted by C in FIG. 2F. As illustrated in FIG. 2F, the point D is the point on the contour of the opening of the hollow cell section positioned at a greater distance from the center of the opening than at least one (here, the point C) of the six points positioned on straight lines coupling the vertices and the center of the opening. With a shape having such points, a range of motion in the bottom surface at the opening side is large, to make the adhesive region easily deformable, making it possible to improve followability to the surface of, for example, skin, and improve close adhesiveness.

More specific examples of the shape of the opening of the hollow cell section include an approximately star shape, a cruciform, and a circular shape.

The size, structure, shape, and material of the flexible hollow structure of the present disclosure are not particularly limited and may be appropriately selected depending on the intended purpose.

The structure of the flexible hollow structure of the present disclosure is not particularly limited and may be appropriately selected depending on the intended purpose so long as the structure includes a plurality of hollow cell sections described above. For example, it is preferable that a plurality of hollow cell sections be arranged adjacently, and that a partitioning wall that is positioned as the circumferential side surface of a hollow cell section to define the hollow cell section and via which a plurality of hollow cell sections are arranged adjacently have cross-sectional areas that are approximately constant in the direction extending from the bottom surface side of the hollow cell section at the other end opposite to the one end toward the opening side.

“A plurality of hollow cell sections being arranged adjacently and the circumferential side surface of a hollow cell section positioned to define the hollow cell section” means that a plurality of hollow cell sections share the partitioning walls of one another and are formed as individual hollow cell sections by being arranged adjacently.

“A partitioning wall via which a plurality of hollow cell sections are arranged adjacently having cross-sectional areas that are approximately constant in the direction extending from the bottom surface side of the hollow cell section at the other end opposite to the one end toward the opening side” means that the thickness of the partitioning wall via which the plurality of hollow cell sections are arranged adjacently is approximately constant throughout the partitioning wall from the bottom surface side until before the opening region.

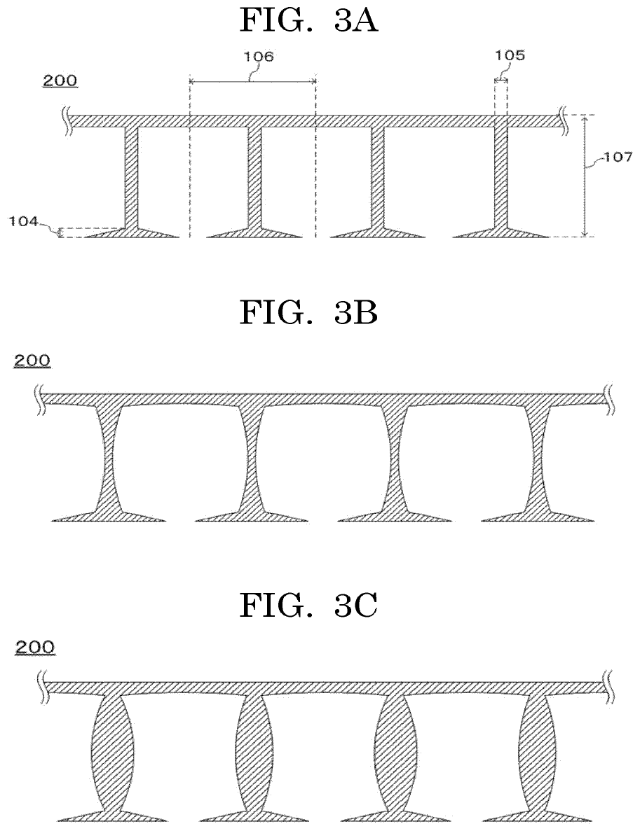

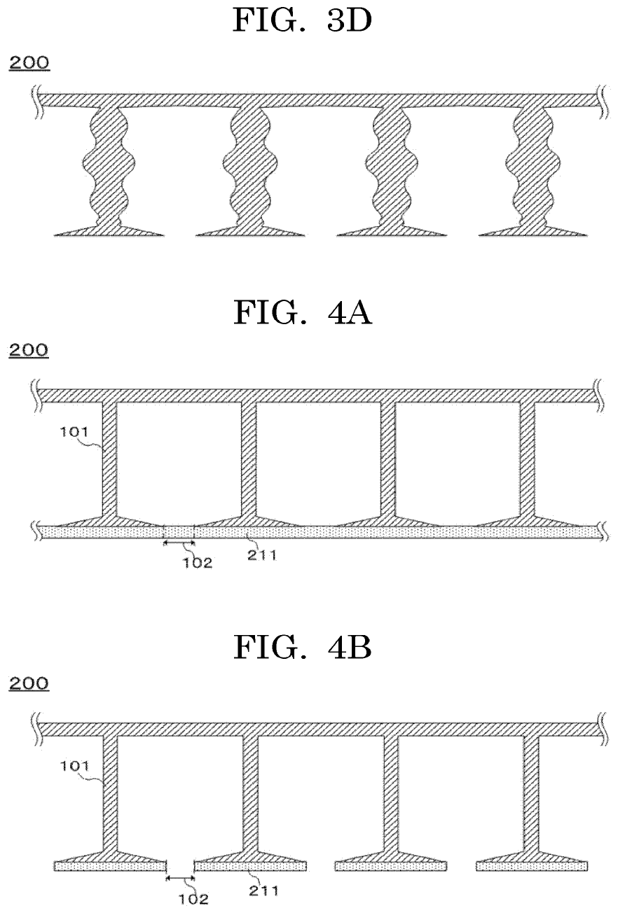

The thickness of a partitioning wall, in which the opening of the hollow cell section is formed, (i.e., the thickness in a direction from the bottom surface of the hollow cell section to the opening, denoted by 104 in FIG. 3A) is not particularly limited, may be appropriately selected depending on the intended purpose, and is preferably 0.1 micrometers or greater but 20 micrometers or less and more preferably 0.1 micrometers or greater but 5 micrometers or less. The shape of the partitioning wall, in which the opening of the hollow cell section is formed (i.e., the shape of a cross-section taken along 10-10′ of FIG. 2B), is preferably a tapered shape that is thinner at positions closer to the contour of the opening. With the partitioning wall, in which the opening of the hollow cell section is formed, having a tapered shape, the flexibility of the partitioning wall, in which the opening is formed, can be improved, making it possible to improve close adhesiveness with, for example, skin.

The thickness of the partitioning wall via which the plurality of hollow cell sections are arranged adjacently (denoted by 105 in FIG. 3A) is not particularly limited, may be appropriately selected depending on the intended purpose, and, for example, is preferably 1 micrometer or greater but 20 micrometers or less and more preferably 1 micrometer or greater but 10 micrometers or less.

The pitch between the hollow cell sections (i.e., the distance between the centers of adjacent hollow cell sections, denoted by 106 in FIG. 3A) is not particularly limited, may be appropriately selected depending on the intended purpose, and, for example, is preferably 50 micrometers or greater but 400 micrometers or less and more preferably 100 micrometers or greater but 300 micrometers or less.

The height of the hollow cell section (i.e., the distance from the bottom surface of the hollow cell section to the opening, denoted by 107 in FIG. 3A) is not particularly limited, may be appropriately selected depending on the intended purpose, and, for example, is preferably 50 micrometers or greater but 800 micrometers or less and more preferably 100 micrometers or greater but 400 micrometers or less.

The material of the flexible hollow structure of the present disclosure is not particularly limited and may be appropriately selected depending on the intended purpose so long as flexibility can be expressed in the hollow structure. For example, materials that are insulators and have a low stimulation or toxicity on living things can be used. Examples of the materials that are insulators and have a low stimulation or toxicity on living things include biocompatible materials, thermoplastic resins, polymeric materials, ultraviolet-ray-curable resins, and polydimethyl siloxane.

Examples of the biocompatible materials include: soluble substances of biological origin such as chitosan, collagen, gelatin, hyaluronic acid (HA), alginic acid, pectin, carrageenan, chondroitin (sulfate), dextran (sulfate), polylysine, carboxymethyl chitin, fibrin, agarose, pullulan, and cellulose; biocompatible substances such as polyvinyl pyrrolidone (PVP), polyethylene glycol (PEG), polyvinyl alcohol (PVA), hydroxypropyl cellulose (HPC), hydroxyethyl cellulose (HEC), hydroxypropyl methyl cellulose (HPMC), sodium carboxymethyl cellulose, polyalcohol, gum arabic, alginate, cyclodextrin, dextrin, dextrose, fructose, starch, trehalose, glucose, maltose, lactose, lactulos, fructose, turanose, melitose, melezitose, dextran, sorbitol, xylitol, palatinit, polylactic acid, polyglycolic acid, polyethylene oxide, polyacrylic acid, polyacrylamide, polymethacrylic acid, and polymaleic acid; derivatives of the substances mentioned above; or mixtures of these substances.

Examples of the thermoplastic resins include: polyolefins such as polyethylene, polypropylene, and ethylene-α-olefin copolymers; polyesters such as polyamide, polyurethane, polyethylene terephthalate, polybutylene terephthalate, polycyclohexane terephthalate, polyethylene, and G-naphthalate; and fluororesins such as PTFE and ETFE.

When a material is one with which it is difficult to form the hollow structure, a surfactant may be used.

Examples of the surfactant include: anionic surfactants such as calcium stearate, magnesium stearate, and sodium lauryl sulfate; cationic surfactants such as benzalkonium chloride, benzethonium chloride, and cetylpyridinium chloride; and nonionic surfactants such as glyceryl monostearate, sucrose fatty acid ester, polyoxyethylene hydrogenated castor oil, and polyoxyethylene sorbitan fatty acid ester.

When a soluble material such as gelatin is used, an insolubilizing agent may added in order to improve water resistance.

Examples of the insolubilizing agent include: organic compounds such as quinones and ketones; and inorganic compounds such as ferric ion and chromium.

The organic compounds preferably have a pH level of around 8. The inorganic compounds preferably have a pH level of around 4.5. Organic compounds are preferable in order not to cause metal allergies during application on skin.

When no insolubilizing agent is introduced, the material can be insolubilized through irradiation with heat or y rays.

Further, adhesiveness-variable materials may also be used in addition to the above-described materials that are insulators and have a low stimulation or toxicity on living things. Use of an adhesiveness-variable material makes it possible to prevent the flexible hollow structure of the present disclosure, when used in, for example, an adhesive tape, from being broken when the adhesive tape is peeled.

Examples of the adhesiveness-variable materials include materials that are solid during application (elongation) of the material and are liquid during peeling, materials that are solid during application (elongation) of the material and are gaseous or liquid during peeling, and materials that have adhesiveness during application (elongation) of the material and have non-adhesiveness during peeling.

Examples of the materials that are solid during application (elongation) of the material and are liquid during peeling include hot-melt adhesives that transform from solids to liquids by heating. When the material that is solid during application (elongation) of the material and is liquid during peeling is a hot-melt adhesive, the hot-melt adhesive that remains in the hollow structure can be used for adhesion to other members.

Examples of the materials that are solid during application (elongation) of the material and are gaseous or liquid during peeling include water (water vapor and ice). When the material that is solid during application (elongation) of the material and is gaseous or liquid during peeling is water, water may be applied over a substrate and cooled to ice with a temperature controlling device before application of the material. This makes it possible to increase close adhesiveness with the material, and after the hollow structure is produced, to heat the ice with the temperature controlling device to liquefy the ice for peeling, and subsequently perform heating for drying. For peeling, it is also possible to heat the ice and vaporize the ice as water vapor.

Examples of the materials that have adhesiveness during application (elongation) of the material and have non-adhesiveness during peeling include materials of which viscoelasticity changes between before and after ultraviolet irradiation, and specifically, the same adhesive materials as used in dicing tapes for preventing chips from being scattered during dicing of a silicon wafer. The materials that have adhesiveness during application (elongation) of the material and have non-adhesiveness during peeling may be cured by ultraviolet irradiation, in order that the materials may be peeled when close adhesiveness of the materials becomes low through curing.

### [Method for Producing Flexible Hollow Structure]

It is possible to refer to the methods described in, for example, Japanese Patent No. 4678731, Japanese Patent No. 4869269, and Japanese Unexamined Patent Application Publication No. 2017-114069, as examples of the method for producing the flexible hollow structure of the present disclosure.

The outline of the method for producing a honeycomb structure described in the Japanese Patent No. 4869269 will be described below.

(1) A base material is positioned over a member having independent concaves (hereinafter, the member may be referred to as “template”) in a manner that the base material covers the concaves. The base material is formed of a material coated over a protective member (e.g., an uncured ultraviolet-ray-curable resin).

(2) The atmosphere surrounding the base material and the template is depressurized (to a vacuum state), in order to relatively generate pressure in the gas in the concaves and simultaneously expand the base material over the concaves by the pressure, to form a hollow structure (honeycomb structure).

(3) When the partitioning walls of the hollow sections have grown to a desired height, the base material is cured by irradiation with energy rays (e.g., ultraviolet rays).

(4) Subsequently, the hollow structure is released from the template.

As the case may be, the following step (5) may be added.

(5) Because the released hollow structure has a shape of being closed at one surface, openings are formed by machining (cutting) to form the hollow sections into through-holes extending from one surface to the other surface.

In the production of the flexible hollow structure of the present disclosure, forming the base material to have a small thickness in the above step (1) and adjusting the absolute pressure to a low level in the above step (2) make it possible to provide the partitioning walls, in which the openings of the hollow cell sections are formed, with a smaller thickness (thickness: the thickness in a direction from the bottom surfaces of the hollow cell sections to the openings) than in existing hollow structures, and to form the partitioning walls into an arbitrary shape.

The height of the hollow cell sections of the flexible hollow structure and the thickness of the partitioning walls via which the plurality of hollow cell sections are arranged adjacently can be controlled based on pressure control during production and adjustment of the mechanical properties (e.g., viscosity, strength, and total elongation) of the base material.

The template has independent concaves. The arrangement of the concaves determines the shape of the hollow cell sections. For example, when the concaves are arranged in a staggered arrangement, the hollow cell sections have a hexagonal shape. When the concaves are arranged in a grid arrangement, the hollow cell sections have a quadrangular shape. The pitch (distance) between the centers of the concaves defines the pitch between the centers of the hollow sections.

Examples of the material of the template include nickel, silicone, stainless steel, and copper.

A bonding device (jig) can be used to bring the base material into close adhesion with the template. Examples of the jig include a jig including a roller member.

It is preferable to perform pressure control in order to prevent the base material from invading the concaves of the template more than needed. It is preferable to bond the base material and the template from an end in order to prevent bubbles from mixing into other regions.

A releasing device (jig) can be used to release the flexible hollow structure from the template. For example, the flexible hollow structure is nipped and pulled up with a tweezers-like jig to be released.

The protective member constituting the base material is a member to be coated with a material, and is used for protection against outgassing in the depressurizing step and for relaxation of stress concentration for protection against chipping in the releasing step. When the protective member is coated with an ultraviolet-ray-curable resin as the material as described above, it is preferable that the protective member be transmissive to the ultraviolet rays with which the protective member is irradiated. Examples of the protective member include protective members formed of flexible plastics such as polyethylene terephthalate (PET) and polyethylene (PE).

### (Adhesive Sheet)

An adhesive sheet of the present disclosure includes the flexible hollow structure of the present disclosure, and an adhesive provided in contact with the openings of the flexible hollow structure, and further includes other members as needed.

The flexible hollow structure of the adhesive sheet of the present disclosure is the same as the flexible hollow structure of the present disclosure. Hence, description of the flexible hollow structure will be skipped.

### —Adhesive—

The adhesive is provided at the opening side of the flexible hollow structure of the present disclosure.

The method for providing the adhesive at the opening side of the flexible hollow structure is not particularly limited and may be appropriately selected depending on the intended purpose. For example, the adhesive dissolved in a solvent may be coated by screen printing, dispenser printing, or coating methods, and then dried. In this way, the adhesive can be formed. A hot-melt film may also be used as the adhesive.

It is preferable that the thickness of the adhesive be less than or equal to twice as large as the shortest distance between the centers of adjoining hollow cell sections. This enables the adhesive to easily deform, allowing expectation for reduction in the adhesive force.

As the adhesive, an adhesive that can provide a sufficient tackifying force (adhesive force) even when formed as a thin layer is preferable.

Examples of the adhesive include natural rubbers, synthetic rubbers/elastomers, vinyl chloride/vinyl acetate copolymers, polyvinyl alkyl ethers, polyacrylate, and modified polyolefin-based resin-based adhesives, or curable adhesives obtained by adding a curing agent such as isocyanate in these adhesives. Among these adhesives, curable adhesives of the types of adhesives used for polyolefin films and polyester films are preferable.

As the adhesive, an adhesive free of a resin is preferable. Examples of the adhesive free of a resin include CMC (carboxymethyl cellulose) of plant-derived polysaccharides. An adhesive free of a resin can reduce stimulation on skin.

The manner for locating the adhesive is not particularly limited and may be appropriately selected depending on the intended purpose. Examples of the manner of locating include a manner of locating the adhesive over the surfaces of the partitioning walls, in which the openings are formed, and a manner of locating the adhesive also inside the hollow cell sections of the flexible hollow structure near the openings.

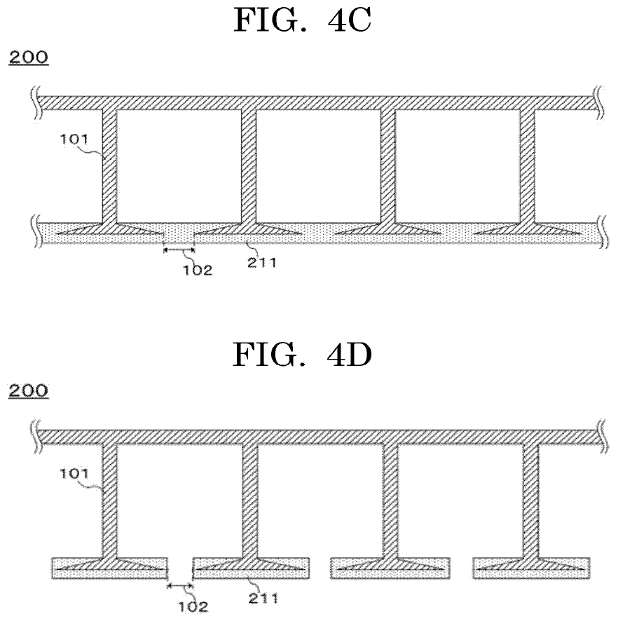

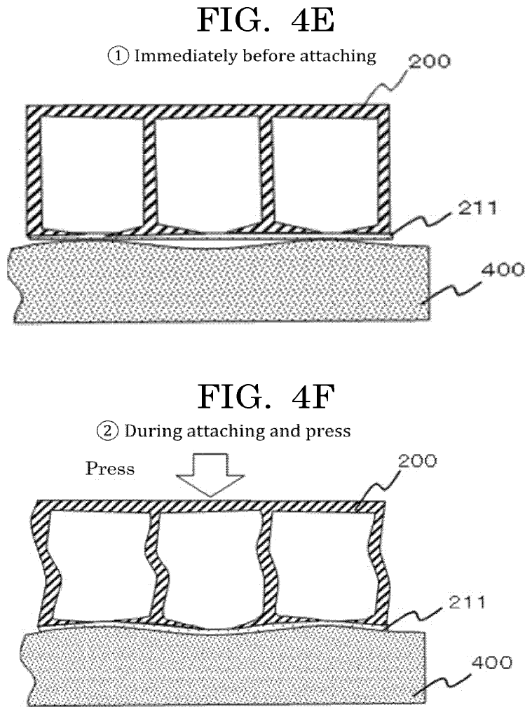

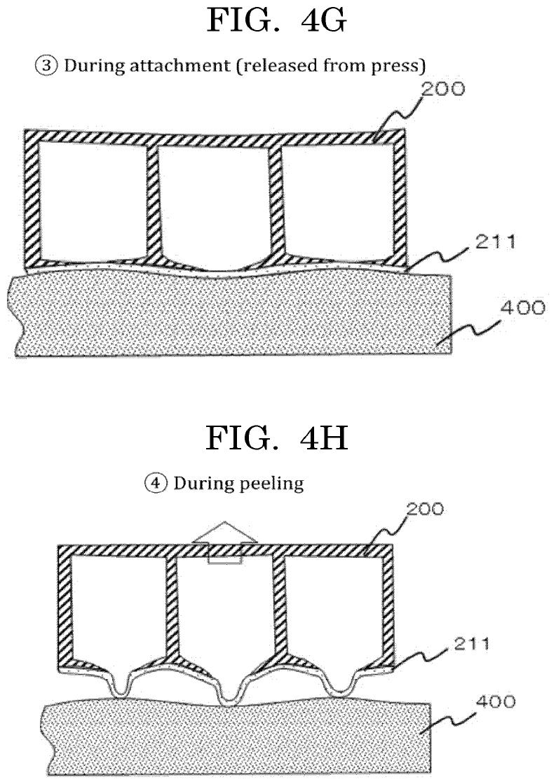

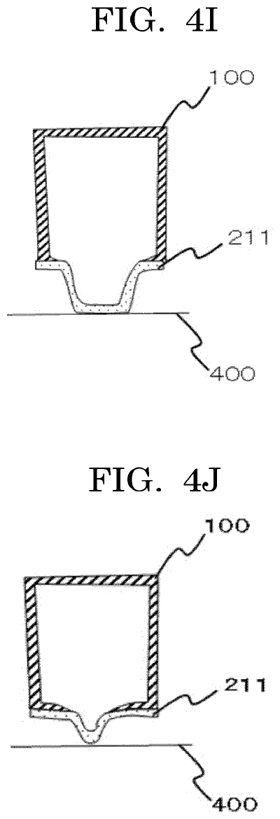

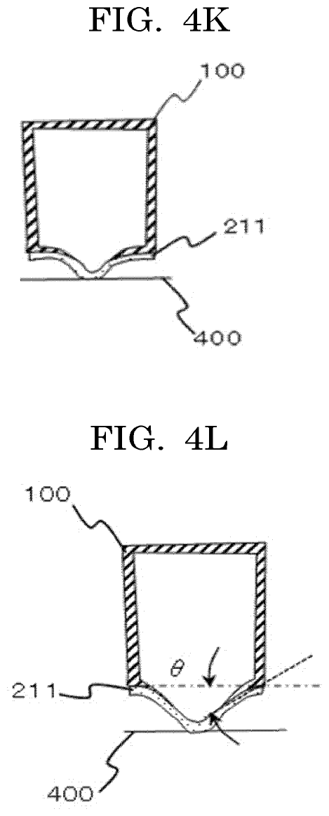

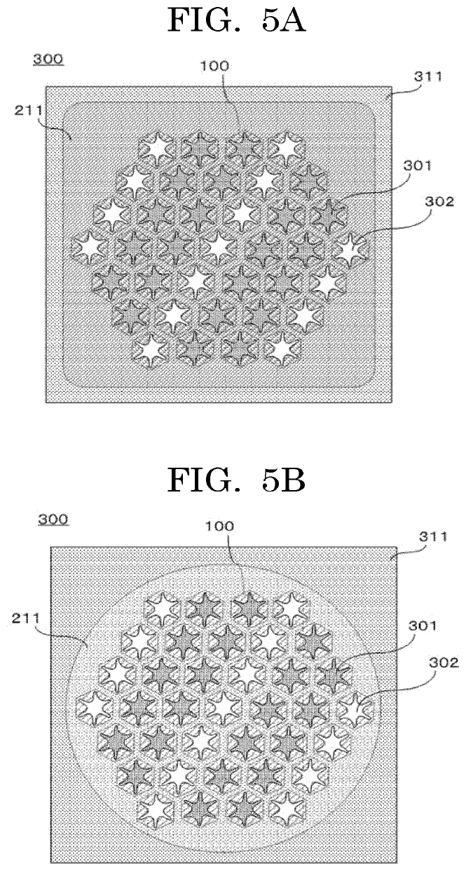

As the manner of locating the adhesive over the surfaces of the partitioning walls, in which the openings are formed, for example, the adhesive may be located in a manner to cover the openings as illustrated in FIG. 4A, or the adhesive may be located only over the partitioning walls, in which the openings are formed, to keep the openings as illustrated in FIG. 4B.

Examples of the manner of locating the adhesive also inside the hollow cell sections of the flexible hollow structure near the openings include, as illustrated in FIG. 4C and FIG. 4D, a manner of locating the adhesive also inside the hollow cell sections in the manner of locating the adhesive illustrated in FIG. 4A and FIG. 4B. The manner of locating the adhesive also inside the hollow cell sections of the flexible hollow structure near the openings provides the adhesive with an anchoring effect to improve close adhesiveness between the partitioning walls and the adhesive. This makes it possible to suppress the adhesive from being detached from the flexible hollow structure during peeling of the adhesive tape.

The manner for locating the adhesive over the adhesive tape of the present disclosure is not particularly limited and may be appropriately selected depending on the intended purpose. For example, the adhesive may be located solidly all over the surface of the flexible hollow structure at the opening side or may be located partially.

It is preferable that any hollow cell sections of the flexible hollow structure free of the adhesive contain air. This makes it possible to distribute and decay, for example, an external impact force and suppress stimulation on skin.

### <Other Members>

Examples of the other members include a support, a stress relaxing layer, a close adhesive layer, an optical adjustment layer, an anti-Newton ring layer, an anti-glare layer, a matting agent layer, a protective layer, an antistatic layer, a smoothing layer, an adhesiveness improving layer, a light shielding layer, an antifog layer, an antifouling layer, and a printed layer.

### —Support—

The support is not particularly limited and may be appropriately selected depending on the intended purpose. Examples of the support include non-woven fabric formed of polyethylene terephthalate (PET) materials, and fabric (kitted work) formed of rayon, polyester, or urethane materials and rich in breathability and stretchability.

Operations when attaching and peeling the adhesive sheet of the present disclosure will further be described with reference to the drawings.

FIG. 4E to FIG. 4I are views illustrating example operations when attaching and peeling the adhesive sheet of the present disclosure. As illustrated in FIG. 4E, when the adhesive sheet is brought into contact with an adherend such as skin, the adhesive of the adhesive sheet and the surface of the skin do not perfectly contact each other due to stiffness of the adhesive sheet. Therefore, as illustrated in FIG. 4F, the bottom surface side of the adhesive sheet is pressed to bring the adhesive of the adhesive sheet into contact with the surface of the skin. Here, because the partitioning walls, in which the openings of the adhesive sheet are formed, can deform in a manner to follow the bumps of the surface of the skin, the adhesive sheet can be kept in adhesion with the skin following the profile of the surface of the skin as illustrated in FIG. 4G even when the adhesive sheet is released from the press. During peeling, as illustrated in FIG. 4H and FIG. 4L, the adhesive peels first at immediately below the partitioning walls that are undergoing less deformation, and the adhesive can gradually peel without concentrating stress on the skin owing to deformation of the partitioning walls, in which the openings are formed, and of the adhesive.

### (Functional Adhesive Sheet)

A functional adhesive sheet of the present disclosure includes the adhesive sheet of the present disclosure, and a functional material provided in the hollow cell sections of the flexible hollow structure of the adhesive sheet, and further includes other members as needed.

The adhesive sheet in the functional adhesive sheet of the present disclosure is the same as the adhesive sheet of the present disclosure.

Hence, description of the adhesive sheet will be skipped.

### —Functional Material—

It is preferable that the functional material be formed of a dispersion liquid of nanoparticles encapsulating an effective component.

The functional materials refer to compositions that contain drugs, quasi-drugs, or cosmetics and can be directly applied to skin and mucous membranes. Specific examples of the functional materials include medicinal cosmetics, nutrients, diagnostic drugs, and therapeutic drugs.

The functional materials need not indispensably be particulate forms, but may be drugs (low-molecular drugs or high-molecular drugs) or cosmetics hitherto used.

The drugs are not particularly limited so long as the drugs are physiologically active substances and have percutaneous absorbability. For example, drugs of the following types can be used: corticosteroids, anti-inflammatory analgestics, hypnotics and sedatives, tranquilizers, antihypertensive agents, hypotensive diuretics, antibiotics, anesthetics, antimicrobials, antifungal drugs, vitamin preparations, coronary vasodilators, antihistamine agents, antitussive agents, sex hormones, antidepressants, cerebral circulation ameliorants, antiemetics, antitumor agents, and biological drugs. Two or more of these drugs may be used in combination as needed.

The content of the drug can be appropriately selected depending on the drug type and the purpose for administration. The content of the drug is preferably from 0.1% by mass through 40% by mass in the percutaneous absorption preparation. When the content of the drug is less than 0.1% by mass, release of a therapeutically effective dose cannot be expected. When the content of the drug is greater than 40% by mass, the therapeutic effect is saturated and there is an economic disadvantage.

In preparations that need a drug replenishing layer as a requisite among the percutaneous absorption preparations, the layer is inserted and maintained in a state of a solution, a dispersion liquid, or gel containing the drug and an absorption aid.

Because of the need for delivering the drug in a high amount into the easily-peelable adhesive layer without fail, it is preferable that the drug be at a saturated concentration in the drug replenishing layer, more preferably at a concentration of from 2 mg/cm² through 80 mg/cm², and yet more preferably at a concentration of from 4 mg/cm² through 60 mg/cm².

The drug may be a free body or a salt.

The drug is not particularly limited and may be appropriately selected depending on the intended purpose. Examples of the drugs include local anesthetics (e.g., bupivacaine hydrochloride and mepivacaine hydrochloride), anticonvulsants (e.g., sodium valproate), painkillers (e.g., morphine hydrochloride, fentanyl citrate, and buprenorphine hydrochloride), antipyretic analgestics (e.g., sulpyrine, antipyrine, and acetaminophen), antipsychotic drugs (e.g., chlorpromazine hydrochloride, levomepromazine hydrochloride, and clocapramine hydrochloride), antidepressants (e.g., imipramine hydrochloride, trazodone hydrochloride, and fluvoxamine maleate), anxiolytics (e.g., diazepam, alprazolam, and tandospirone citrate), tranquilizers (e.g., hydroxyzine hydrochloride), cerebral function activators (e.g., tiapride hydrochloride and protirelin tartrate), cerebral circulation ameliorants (e.g., isosorbide, pentoxifylline, and fasudil hydrochloride), Parkinson's disease remedies (e.g., benserazide hydrochloride, amantadine hydrochloride, and talipexol hydrochloride), muscle relaxants (e.g., eperisone hydrochloride, tizanidine hydrochloride, and tolperisone hydrochloride), peptic ulcer agents (e.g. scopolamine butylbromide, pirenzepine hydrochloride, and timepidium bromide), antihistamine agents (e.g., chlorpheniramine maleate, promethazine hydrochloride, and cetirizine hydrochloride), chemical transmitter release inhibitors (e.g., emedastine fumarate, suplatast tosylate, and epinastine hydrochloride), heart disease therapeutic agents (e.g., aminophylline, diltiazem hydrochloride, nicorandil, propranolol hydrochloride, isoprenaline hydrochloride, disopyramide phosphate, and procainamide hydrochloride), antihypertensive drugs (e.g., captopril, enalapril maleate, amosulalol hydrochloride, prazosin hydrochloride, urapidil, and clonidine hydrochloride), vasodilators (e.g., trazoline hydrochloride), vasoconstrictors (e.g., amezinium metilsulfate, ethylephrine hydrochloride, phenylephrine hydrochloride, and midodrine hydrochloride), antihyperlipidemic drugs (e.g., pravastatin sodium, fluvastatin sodium, and cerivastatin sodium), antitussive and expectorant drugs (e.g., dextromethorphan hydrobromide, hominoben hydrochloride, and acetylcysteine), antasthmatic drugs (e.g., clenbuterol hydrochloride, fenoterol hydrobromide, and procaterol hydrochloride), H2 blockers (e.g., ranitidine hydrochloride and roxatidine acetate hydrochloride), proton pump inhibitors (e.g., omeprazole, lansoprazole, and rabeprazole), antiemetics (e.g., granisetron hydrochloride, azasetron hydrochloride, ondansetron hydrochloride, and ramosetron hydrochloride), non-steroidal anti-inflammatory agents (e.g., loxoprofen sodium, flurbiprofen, diclofenac sodium, and tiaramide hydrochloride), anti-rheumatic drugs (e.g., bucillamine and penicillamine), urologic disease drugs (e.g., oxybutynin hydrochloride, tamsulosin hydrochloride, and propiverine hydrochloride), and β-blockers (e.g., bisoprolol fumarate and betaxolol hydrochloride).

Moreover, any substances can be used so long as the substances have fluidity and are suitable for other purposes such as medical care, cosmetics, and agriculture. Examples of the substances include medicinal cosmetics, nutrients, diagnostic drugs, and therapeutic drugs.

For example, the drugs effective as drugs that can be introduced in the present disclosure may be impregnated with cosmetic materials or medicinal components, particularly high-molecular medicinal components.

Examples of the cosmetic materials include: skin whitening ingredients such as ascorbic acid, vitamin C ethyl, vitamin C glycoside, ascorbyl palmitate, kojic acid, rucinol, tranexamic acid, oil-soluble licorice extracts, vitamin A derivatives, and placenta extracts; anti-wrinkle ingredients such as retinol, retinoic acid, retinol acetate, retinol palmitate, EGF, cell culture extracts, and acetylglucosamine; blood circulation promoting ingredients such as tocopherol acetate, capsaicin, and nonyl acid vanillylamide: weight control ingredients such as Raspberry ketone, evening primrose extracts, and seaweed extracts; antimicrobial ingredients such as isopropyl methyl phenol, photosensitizers, and zinc oxide; vitamins such as vitamin D2, vitamin D3, and vitamin K; and saccharides such as glucose, trehalose, and maltose.

Examples of the high-molecular medicinal components include fragments of biologically active peptides and derivatives of biologically active peptides, nucleic acids, oligonucleotides, various antigenic proteins, bacteria, and viruses.

Examples of the dispersion medium of the dispersion liquid of the nanoparticles include water, electrolyte aqueous solutions, and organic solvents. Water and electrolyte aqueous solutions are preferable, and electrolyte aqueous solutions are more preferable.

The electrolytes are not particularly limited and may be appropriately selected depending on the intended purpose so long as the electrolytes are biocompatible materials. Examples of the electrolytes include sodium chloride, potassium chloride, sodium bromide, potassium bromide, calcium chloride, and calcium bromide.

The kind of the dispersion medium can be appropriately selected depending on the kind of the nanoparticles and depending on the body parts to which the nanoparticles are delivered (e.g., stratum corneum, dermic layer, and blood).

### —Nanoparticles—

The average particle diameter of the nanoparticles is preferably 500 nm or less, more preferably from 10 nm through 100 nm, and yet more preferably from 40 nm through 80 nm.

When the particle diameter of the nanoparticles is less than 10 nm, the nanoparticles have a high skin permeability but may not be able to exhibit the desired effect because of diffusion. On the other hand, when the particle diameter of the nanoparticles is greater than 100 micrometers, the nanoparticles may have a low skin permeability because the nanoparticles are greater than the size of the skin pores.

The nanoparticles are not particularly limited so long as the nanoparticles can be electrophoresed while encapsulating the desired effective component. Examples of the nanoparticles include liposomes, micelles, and organic nanotubes.

The materials for constituting the nanop articles may be any biocompatible materials that can form nanosized liposomes and micelles. Examples of the materials include polyparadioxanone (PPDX), polylactide-co-glycolide (PLGA), polycaprolactone, polylactic acid, polyanhydrides, polyorthoesters, polyether esters, polyester amides, polyamide, polyethylene glycol, and polybutyric acid.

The concentration of the nanop articles can be appropriately selected depending on the kind of the effective component. For example, the concentration of the effective component encapsulated is preferably in the range of from 0.1 mM through 100 mM. A concentration higher than 100 mM is difficult to produce.

Setting the concentration of the nanoparticles within the range of from 0.1 mM through 100 mM enables the desired effect. For example, the concentration of the nanoparticles is preferably from 0.5 mM through 15 mM, more preferably from 1 mM through 10 mM, and yet more preferably from 1 mM through 7 mM.

### —Effective Component—

The effective component encapsulated in the nanoparticles is not particularly limited and may be appropriately selected depending on the intended purpose. Examples of the effective component include medicinal components (particularly, high-molecular medicinal components) as the materials for medicinal cosmetics. Specific examples of the effective component include: skin whitening ingredients such as ascorbic acid, vitamin C ethyl, vitamin C glycoside, ascorbyl palmitate, kojic acid, rucinol, tranexamic acid, oil-soluble licorice extracts, vitamin A derivatives, and placenta extracts; anti-wrinkle ingredients such as retinol, retinoic acid, retinol acetate, retinol palmitate, EGF, cell culture extracts, and acetylglucosamine; blood circulation promoting ingredients such as tocopherol acetate, capsaicin, and nonyl acid vanillylamide: weight control ingredients such as Raspberry ketone, evening primrose extracts, and seaweed extracts; antimicrobial ingredients such as isopropyl methyl phenol, photosensitizers, and zinc oxide; vitamins such as vitamin D2, vitamin D3, and vitamin K; and saccharides such as glucose, trehalose, and maltose.

Examples of the high-molecular medicinal components include fragments of biologically active peptides and derivatives of biologically active peptides, nucleic acids, oligonucleotides, various antigenic proteins, bacteria, and viruses.

In order to promote percutaneous absorbability, the following substances may be added: nonionic surfactants such as glyceryl monostearate and sucrose fatty acid esters; water-soluble high-molecular compounds such as carboxylic acid; water-soluble chelate agents such as EDTA; aromatic carboxylic acid compounds such as salicylic acid and derivatives of salicylic acid; aliphatic carboxylic acid compounds such as capric acid and oleic acid; bile salts; propylene glycol; hydrogenated lanolin; isopropyl myristate; diethyl sebacate; urea; lactic acid; and atone.

Moreover, ultraviolet absorbers or ultraviolet scattering agents may be encapsulated in the nanop articles.

Examples of the ultraviolet absorbers include: cinnamic acid-based ultraviolet absorbers such as octyl cinnamate, ethyl-4-isopropyl cinnamate, methyl-2,5-diisopropyl cinnamate, ethyl-2,4-diisopropyl cinnamate, methyl-2,4-diisopropyl cinnamate, propyl-p-methoxycinnamate, isopropyl-p-methoxycinnamate, isoamyl-p-methoxycinnamate, octyl-p-methoxycinnamate, 2-ethoxyethyl-p-methoxycinnamate, cyclohexyl-p-methoxycinnamate, ethyl-α-cyano-β-phenyl cinnamate, 2-ethylhexyl-α-cyano-β-phenyl cinnamate, and glycerylmono-2-ethylhexanoyl-diparamethoxycinnamate; benzophenone-based ultraviolet absorbers such as 2,4-dihydroxybenzophenone, 2,2′-dihydroxy-methoxybenzophenone, 2,2′-dihydroxy-4,4′-diemethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl-4′-phenyl-benzophenone-2-carboxylate, 2-hydroxy-4-n-octoxybenzophenone, and 4-hydroxy-3-carboxybenzophenone; paraaminobenzoic acid-based ultraviolet absorbers such as PABA monoglycerin ester, N,N-dipropoxy PABA ethyl ester, N,N-diethoxy PABA ethyl ester, N,N-dimethyl PABA ethyl ester, N,N-dimethyl PABA butyl ester, and N,N-dimethyl PABA methyl ester; salicylic acid-based ultraviolet absorbers such as amyl salicylate, menthyl salicylate, homomenthyl salicylate, octyl salicylate, phenyl salicylate, benzyl salicylate, and p-isopropanol phenyl salicylate; ultraviolet absorbers such as 3-(4′-methyl benzylidene)-d-camphor, 3-benzylidene-d, 1-campphor, urocanic acid, urocanic acid ethyl ester, octyl triazone, and 4-methoxy-4′-t-butyl benzoyl methane.

Examples of the ultraviolet scattering agents include titanium oxide particles, zinc oxide, and cerium oxide.

As other components, additive components commonly blended in functional materials may be encapsulated. The kinds and amounts of the other components may be appropriately selected, provided that the stability of the composition and the desired effect of the effective component are not spoiled.

Examples of the additive components include: nonionic polymers such as guar gum and tamarind gum; cationic polymers such as cationized cellulose and diallyl dimethyl ammonium chloride polymers; anionic polymers such as xanthan gum and sodium alginate; and other natural water-soluble compounds or derivatives of natural water-soluble compounds, surfactants, oils, colorants, antiseptics, chelate agents, antioxidants, humectants, lower alcohols, polyvalent alcohols, fragrances, tonics, and pH adjustors.

The surfactants are not particularly limited and are added for emulsification, solubilization, and dispersion. Examples of the surfactants include: nonionic surfactants such as POE fatty acid ester, polyglycerin fatty acid esters, POE higher alcohol ethers, and POE or POP block polymers; anionic surfactants such as fatty acid potassium, fatty acid sodium, higher alkyl sulfuric acid ester salts, alkyl ether sulfuric acid ester salts, acyl sarcosinates, and sulfosuccinates; cationic surfactants such as alkyl trimethyl ammonium salts, dialkyl dimethyl ammonium salts, alkyl pyridinium salts, and benzalkonium chloride; and imidazoline-based and betaine-based amphoteric surfactants.

Examples of the oils include: vegetable oils such as olive oil, jojoba oil, castor oil, rice bran oil, and palm oil; animal oils such as squalene, beef tallow, and lanolin; synthetic oils such as silicone oil, polyisobutene, fatty acid esters, and fatty acid glycerin; waxes such as beeswax, Japan tallow, candelilla wax, and carnauba wax; hydrocarbons such as liquid paraffin, ceresin, microcrystalline wax, and vaseline; higher alcohols such as cetanol, stearyl alcohol, and octyl dodecanol; higher fatty acids such as stearic acid, lauric acid, myristic acid, and oleic acid; and silicone resins, silicone rubbers, polyether-modified silicones, and perfluoroethers.

Examples of the colorants include organic dyes and natural dyes such as Blue No. 1, Green No. 3, Red No. 202, Red No. 227, Yellow No. 4, chlorophyll, and β-carotene.

Examples of the humectants include vitamins A, B, C, and E or derivatives of vitamins A, B, C, and E, various amino acids, sodium hyaluronate, and trimethyl glycine.

### <Other Members>

The other members are not particularly limited and may be appropriately selected depending on the intended purpose. Examples of the other members include variable-fluidity materials.

### —Variable-Fluidity Material—

The variable-fluidity material is a material that deforms in response to a low stress. Specific examples of the variable-fluidity material include gases, liquids, or fine solid powders.

For example, when a gas, a liquid, or a fine solid powder is located in the space inside the hollow cell sections as the variable-fluidity material, it is optional whether to provide or not to provide the flexible hollow structure with a bottom surface.

When the flexible hollow structure is provided with a bottom surface, filling the whole space inside the cells with a liquid or a fine solid powder reduces deformability of the hollow cell sections. Hence, the liquid or the fine solid powder is located in part of the space inside the hollow cell sections. Locating the liquid or the fine solid powder in the space inside the hollow cell sections in this way can impart a certain stiffness and reduce breakage during transportation. Moreover, when provided, the bottom surface can prevent contamination with, for example, bacteria from outside.

Furthermore, addition of an antibacterial drug in the liquid and the fine solid powder serving as the variable-fluidity materials can prevent contamination with, for example, bacteria. Use of water-repellent materials as the liquid and fine solid powder can prevent invasion of water during use in, for example, a bathroom.

When no bottom surface is provided, the hollow cell sections opened at both ends function as gas permeable regions C and link to the outside, leading to improvement of perspiration and reduction of skin dampening. This reduces stimulation on skin.

Examples of the variable-fluidity material include: gases such as carbon dioxide (having a vasodilator effect); liquids such as water-soluble gelatinous gelatin, water-repellent vaseline, and glycerin; and solid powders such as gelatin having an average particle diameter of about 50 micrometers, silica, plastics (e.g., PVA and polystyrene), and activated carbon.

Next, an example method for producing the flexible hollow structure in which the hollow cell sections are filled with the functional material and the variable-fluidity material will be described.

The flexible hollow structure 200 illustrated in FIG. 3A is produced according to the method described in Japanese Patent No. 4678731. The flexible hollow structure 200 has a bottom surface, and each hollow cell section has one opening.

In Examples of the present disclosure described below, a shape in which cells have two openings formed by cutting the partitioning walls of the hollow cell section in parallel with the bottom surface with a long blade was used.

Because of having no bottom surface, a flexible hollow structure having no bottom surface has a lower stiffness, is more deformable, and enables a better distribution of a force than a structure having a bottom surface. During use, the flexible hollow structure having no bottom surface can reduce stimulation on skin through relaxation of a stress (e.g., rubbing by clothes due to, for example, bending exercises) to lateral sides. During peeling, the flexible hollow structure having no bottom surface can reduce the adhesive force.

The manner for locating the hollow cell sections containing the functional material in the flexible hollow structure of the functional adhesive sheet is not particularly limited and may be appropriately selected depending on the intended purpose. Examples of the manner include a manner of filling the hollow cell sections having openings provided with the adhesive with the functional material, and a manner of filling the hollow cell sections having openings provided with no adhesive with the functional material. The manner of filling the hollow cell sections having openings provided with the adhesive with the functional material can improve close adhesiveness with an adherend such as skin. The manner of filling the hollow cell sections having openings provided with no adhesive with the functional material enables an efficient release of the functional material because no adhesive is provided at the openings of the hollow cell sections filled with the functional material.

In this case, the manner for locating the adhesive over the functional adhesive sheet of the present disclosure is not particularly limited and may be appropriately selected depending on the intended purpose. For example, the adhesive may be located solidly all over the surface of the flexible hollow structure at the opening side or may be located partially.

When locating the adhesive partially, a manner of locating the functional material in some of the plurality of hollow cell sections of the hollow flexible structure and locating the adhesive in contact with the openings of the hollow cell sections in which the functional material is not located is preferable. That is, it is preferable to selectively locate the adhesive over the partitioning walls, in which the openings of the hollow cell sections not filled the functional material are formed.

The functional adhesive sheet of the present disclosure will now be described with reference to the drawings.

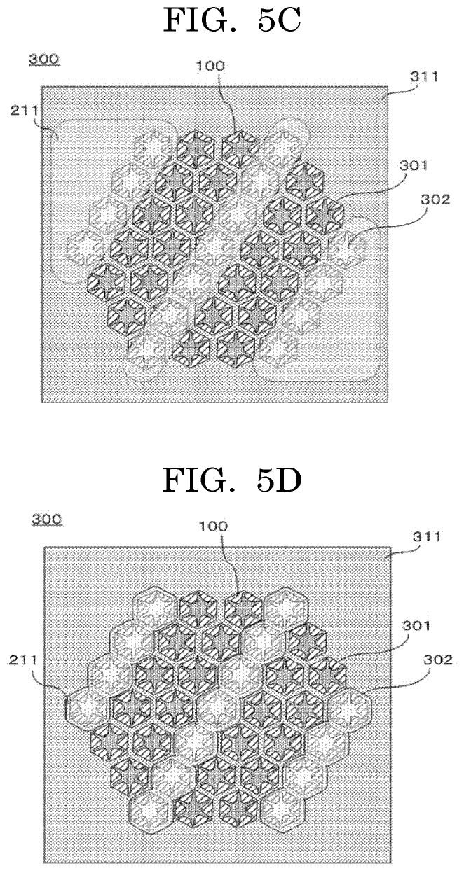

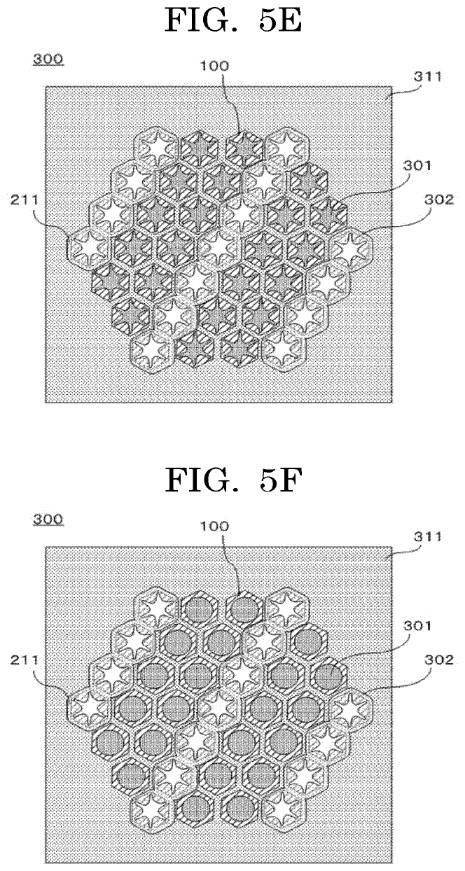

FIG. 5A to FIG. GB are views illustrating examples of the functional adhesive sheet of the present disclosure.

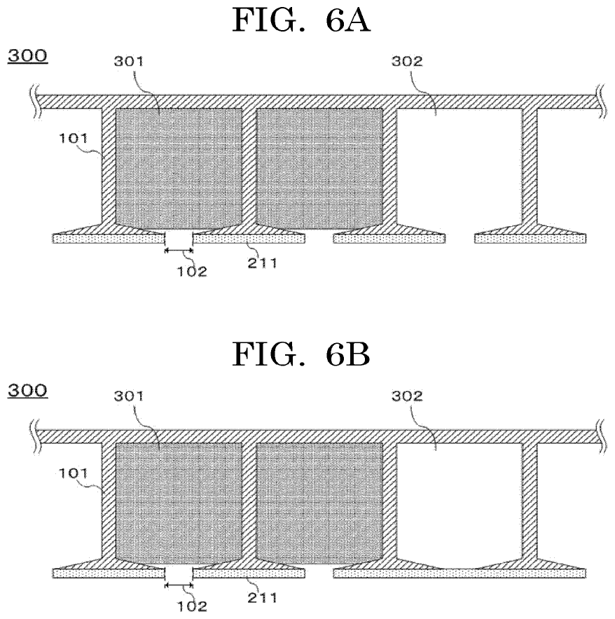

As illustrated in FIG. 5A, the functional adhesive sheet includes a base material 311, hollow cell sections 100 constituting a flexible hollow structure, and an adhesive 211. The functional adhesive sheet 300 illustrated in FIG. 5A includes hollow cell sections filled with a functional material 301 and hollow cell sections 302 filled with no substance. In the functional adhesive sheet 300 illustrated in FIG. 5A, the openings of the hollow cell sections 100 are not blocked with the adhesive 211 as illustrated in FIG. 6A. The adhesive 211, which is chamfered and rounded, has a shape that is preventive against stress concentration and does not easily peel from an adherend such as skin.

FIG. 5B is not different from FIG. 5A except that the shape of the adhesive 211 is changed to a circular shape unlike in FIG. 5A. The adhesive having the circular shape is more unsusceptible to stress and can improve the adhesive force.

FIG. 5C to FIG. 5F are views illustrating examples in which the adhesive 211 is applied in a manner to cover the partitioning walls, in which the openings of the hollow cell sections 100 not filled the functional material 301 are formed. The adhesive 211 may be applied in a manner to block the openings and also cover regions other than the hollow cell sections 100 as illustrated in FIG. 5C, or the adhesive 211 may be applied in a manner to cover only the hollow cell sections 100 not filled with the functional material 301 as illustrated in FIG. 5D.

When an adhesive 211 having a sufficiently high adhesive force is used as illustrated in FIG. 5E, it is possible to opt for a manner of applying the adhesive only over the partitioning walls, in which the openings of the hollow cell sections 100 not filled with the functional material 301 are formed, so as not to block the openings.

FIG. 5F illustrates an example in which the openings of the hollow cell sections 100 filled with the functional material 301 have a circular shape. Forming the openings of the hollow cell sections 100 filled with the functional material 301 in a shape different from the openings of the hollow cell sections 100 not filled with the functional material 301 as illustrated in FIG. 5F makes it possible to provide an opening shape suitable for release of the functional material without spoiling the effects of the present disclosure.

### EXAMPLES

The present disclosure will be described below by way of Examples. The present disclosure will not be construed as being limited to these Examples.

### Comparative Example 1

### Production Example 1

### [Production of Flexible Hollow Structure]

Using an acrylic-based ultraviolet-ray-curable resin (ultraviolet-ray-curable urethane acrylate) as a material, a flexible hollow structure was produced using a template molded and transferred from a patterned mold the according to the method for producing a honeycomb structure described in Japanese Patent No. 4869269.

The dimensions of each part of the flexible hollow structure were as follows.
Area X of a cross-section of each hollow cell section positioned closely to the opening and approximately in parallel with the opening: see Table 1
Opening area: see Table 1
Thickness of partitioning walls via which a plurality of hollow cell sections were arranged adjacently: 5 (micrometers)
Thickness of a partitioning wall, in which the opening of each hollow cell section was formed (thickness in a direction from the bottom surface of each hollow cell section to the opening): no partitioning wall
Pitch between hollow cell sections (center-to-center distance): 200 (micrometers)
Size: 20 mm in longitude x 20 mm in latitude

### [Production of Functional Adhesive Sheet]

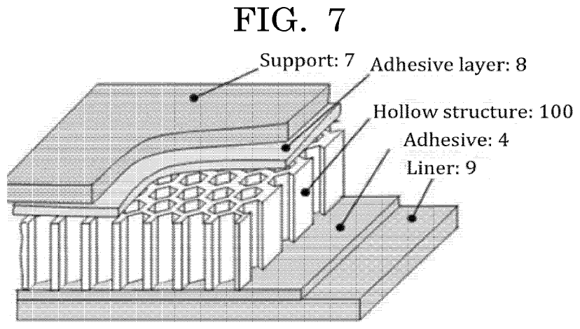

An adhesive (product name: DURO-TAK 87-2516, available from National Starch & Chemicals Ltd.) in which hyaluronic acid serving as a functional material was added by kneading at a final concentration of 20% by mass was diluted two-fold with an organic solvent (ethyl acetate). In the resultant dilute solution, the flexible hollow structure produced was immersed down to the internal side of the partitioning walls, in which the openings of the flexible hollow structure were formed, and dried at a temperature of 80 degrees C. at a relative humidity of 10 w %. The dilute solution was further applied by slit coating over the partitioning walls, in which the openings of the flexible hollow structure were formed, and subsequently dried at a temperature of 80 degrees C. at a relative humidity of 10 w %, to produce a functional adhesive sheet 1 in which the adhesive was provided at the openings of the flexible hollow structure as illustrated in FIG. 7.

### Comparative Example 2

### Production Example 2

A flexible hollow structure, an adhesive sheet, and a functional adhesive sheet 2 were produced in the same manner as in Comparative Example 1, except that unlike in Comparative Example 1, “Area X” and “Opening area” were changed as presented in Table 1.

### Comparative Examples 3 and 4

### Production Examples 3 and 4

Flexible hollow structures and adhesive sheets 1′ and 2′ were produced in the same manner as in Comparative Example 1, except that unlike in Comparative Example 1, no functional material was added in the adhesive, and “Area X” and “Opening area” were changed as presented in Table 1.

### Example 1

### Production Example 5

### [Production of Flexible Hollow Structure]

Using an acrylic-based ultraviolet-ray-curable resin (ultraviolet-ray-curable urethane acrylate) as a material, a flexible hollow structure was produced using a template molded and transferred from a patterned mold the according to the method for producing a honeycomb structure described in Japanese Patent No. 4869269.

The dimensions of each part of the flexible hollow structure were as follows.
Area X of a cross-section of each hollow cell section positioned closely to the opening and approximately in parallel with the opening: see Table 1
Opening area: see Table 1
Thickness of partitioning walls via which a plurality of hollow cell sections were arranged adjacently: 5 (micrometers)
Thickness of a partitioning wall, in which the opening of each hollow cell section was formed (thickness in a direction from the bottom surface of each hollow cell section to the opening): see Table 1
Pitch between hollow cell sections (center-to-center distance): 200 (micrometers)
Size: 20 mm in longitude x 20 mm in latitude

### [Production of Functional Adhesive Sheet]

By a dipping method, the hollow cell sections of the flexible hollow structure produced were filled with hyaluronic acid serving as a functional material in the manner of location as illustrated in FIG. 5F.

Next, an adhesive (product name: DURO-TAK 87-2516, available from National Starch & Chemicals Ltd.) was diluted two-fold with an organic solvent (ethyl acetate). The resultant dilute solution was applied by slit coating over the partitioning walls, in which the openings of the flexible hollow structure were formed, and subsequently dried at a temperature of 80 degrees C. at a relative humidity of 10 w %, to produce a functional adhesive sheet 3 in which the adhesive was provided at the openings of the flexible hollow structure.

### Examples 2 and 3

### Production Examples 6 and 7

Flexible hollow structures, adhesive sheets, and functional adhesive sheets 4 and 5 were produced in the same manner as in Example 1, except that unlike in Example 1, “Area X” and “Opening area” were changed as presented in Table 1.

Next, the functional adhesive sheets and adhesive sheets produced were attached over an adherend 400, which was a silicone rubber having a surface with the condition described below (a silicone rubber obtained by mixing and curing SIM-260 and CAT-260 available from Shin-Etsu Chemical Co., Ltd.), and subsequently peeled under the conditions described below. The adhesive sheets peeled were evaluated according to the evaluation criteria described below.

### <Surface Condition of Adherend>

Surface roughness (Ra): 10 micrometers

### <Peeling Conditions>

The adhesive sheet was folded back by 180 degrees, and while both ends (sides) of the adherend were secured, peeled with an end (one side) of the adhesive sheet picked.
Peeling speed: about 5 mm/sec

### <Presence or Absence of Residual Adhesive Over Silicone Rubber>

A residual adhesive area (mm²) of the adherend was measured by microscopic observation.

TABLE 1

| | | | | | | | | | Evaluation result | |
|---|---|---|---|---|---|---|---|---|---|---|
| | | | Functional | Flexible hollow structure | | | | Location of | Presence or | Tack force |
| | | Adhesive | adhesive | | Area X | Opening area | | functional | absence of | (inclined |
| | | sheet No. | sheet No. | Material | (mm²) | Ratio | (mm²) | material | residual adhesive | ball tack test) |
| Comp. | 1 | — | 1 | Acrylic | 0.03 | 0.1X | 0.003 | Adhesive | 0 | 2 |
| Ex. | | | | ultraviolet-curable | | | | layer | | |
| | | | | resin | | | | | | |
| | 2 | — | 2 | Acrylic | 0.03 | 0.8X | 0.025 | Adhesive | 50 | 3 |
| | | | | ultraviolet-curable | | | | layer | | |
| | | | | resin | | | | | | |
| | 3 | 1′ | — | Acrylic | 0.03 | 0.1X | 0.003 | — | 0 | 3 |
| | | | | ultraviolet-curable | | | | | | |
| | | | | resin | | | | | | |
| | 4 | 2′ | — | Acrylic | 0.03 | 0.8X | 0.025 | — | 60 | 5 |
| | | | | ultraviolet-curable | | | | | | |
| | | | | resin | | | | | | |
| Ex. | 1 | — | 3 | Acrylic | 0.03 | 0.2X | 0.006 | Hollow | 0 | 4 |
| | | | | ultraviolet-curable | | | | cell | | |
| | | | | resin | | | | section | | |
| | 2 | — | 4 | Acrylic | 0.03 | 0.4X | 0.013 | Hollow | 10 | 4 |
| | | | | ultraviolet-curable | | | | cell | | |
| | | | | resin | | | | section | | |
| | 3 | — | 5 | Acrylic | 0.03 | 0.7X | 0.022 | Hollow | 30 | 5 |
| | | | | ultraviolet-curable | | | | cell | | |
| | | | | resin | | | | section | | |

The functional adhesive sheets of Comparative Examples 1 and 2 with a functional material added in the adhesive had a low adhesive force and a low strength. From the results of Comparative Examples 3 and 4, it turned out that a larger opening led to a higher tack force (close adhesiveness during attachment), but to a higher amount of the residual adhesive. As compared, an opening area set to 0.2X or greater but 0.7X or less with respect to the area X succeeded in suppressing the amount of the residual adhesive while improving the tack force (close adhesiveness during attachment). Moreover, the materials used and the structure of the flexible hollow structure enabled functional adhesive sheets excellent in water permeability.

Aspects of the present disclosure are, for example, as follows.

<1> A flexible hollow structure,

wherein the flexible hollow structure has a plurality of hollow cell sections each having an opening at one end, and

wherein when an area of a cross-section of each hollow cell section positioned closely to the opening and approximately in parallel with the opening is X (mm²), an opening area of the opening is 0.2X or greater but 0.7X or less.

<2> The flexible hollow structure according to <1>,

wherein cross sections of each hollow cell section positioned approximately in parallel with the opening have an approximately constant area.

<3> The flexible hollow structure according to <1> or <2>,

wherein the plurality of hollow cell sections are arranged adjacently, and

wherein a partitioning wall that is positioned as a circumferential side surface of each hollow cell section to define the hollow cell section and via which the plurality of hollow cell sections are arranged adjacently has cross-sectional areas that are approximately constant in a direction extending from a bottom surface of each hollow cell section positioned at another end opposite to the one end toward the opening.

<4> The flexible hollow structure according to any one of <1> to <3>,

wherein at least two points on a contour of the opening of each hollow cell section are positioned at different distances from a center of the opening.

<5> The flexible hollow structure according to any one of <1> to <4>,

wherein a contour of the opening of each hollow cell section has an inflection point, and a contour portion at which the inflection point is present is a curve.

<6> The flexible hollow structure according to any one of <1> to <5>,

wherein the cross-section of each hollow cell section positioned approximately in parallel with the opening of the hollow cell section has a honeycomb shape (hexagonal shape).

<7> The flexible hollow structure according to <6>,

wherein when the opening of each hollow cell section is seen in a plan-view perspective, the contour of the opening forms a shape that has six points positioned on straight lines coupling vertices of the honeycomb shape to the center of the opening and a point positioned at a greater distance from the center of the opening than at least one of the six points.

<8> The flexible hollow structure according to any one of <1> to <7>,

wherein when the opening of each hollow cell section is seen in a plan-view perspective, the opening has an approximately star shape.

<9> An adhesive sheet including:

the flexible hollow structure according to any one of <1> to <8>; and

an adhesive provided in contact with the opening of the flexible hollow structure.

<10> The adhesive sheet according to <9>,

wherein the adhesive is also provided inside each hollow cell section of the flexible hollow structure near the opening.

<11> A functional adhesive sheet including:

the adhesive sheet according to <9> or <10>; and

a functional material provided in each hollow cell section of the flexible hollow structure of the adhesive sheet.

<12> The functional adhesive sheet according to <11>,

wherein the functional material is a drug.

<13> The functional adhesive sheet according to <11> or <12>,

wherein the functional material is provided in some of the plurality of hollow cell sections of the flexible hollow structure, and

wherein the adhesive is provided in contact with the opening of each hollow cell section that is not provided with the functional material.

The flexible hollow structure according to any one of <1> to <8>, the adhesive sheet according to any one of <9> to <11>, and the functional adhesive sheet according to <12> or <13> can solve the various problems in the related art and achieve the object of the present disclosure.

What is claimed is:

1. A flexible hollow structure,
wherein the flexible hollow structure has a plurality of hollow cell sections each having an opening at one end, and
wherein when an area of a cross-section of each hollow cell section positioned closely to the opening and approximately in parallel with the opening is X (mm²), an opening area of the opening is 0.2X or greater but 0.7X or less.

2. The flexible hollow structure according to claim 1,
wherein cross sections of each hollow cell section positioned approximately in parallel with the opening have an approximately constant area.

3. The flexible hollow structure according to claim 1,
wherein the plurality of hollow cell sections are arranged adjacently, and
wherein a partitioning wall that is positioned as a circumferential side surface of each hollow cell section to define the hollow cell section and via which the plurality of hollow cell sections are arranged adjacently has cross-sectional areas that are approximately constant in a direction extending from a bottom surface of each hollow cell section positioned at another end opposite to the one end toward the opening.

4. The flexible hollow structure according to claim 1,
wherein at least two points on a contour of the opening of each hollow cell section are positioned at different distances from a center of the opening.

5. The flexible hollow structure according to claim 1,
wherein a contour of the opening of each hollow cell section has an inflection point, and a contour portion at which the inflection point is present is a curve.

6. The flexible hollow structure according to claim 1,
wherein the cross-section of each hollow cell section positioned approximately in parallel with the opening of the hollow cell section has a honeycomb shape (hexagonal shape).

7. The flexible hollow structure according to claim 6,
wherein when the opening of each hollow cell section is seen in a plan-view perspective, a contour of the opening forms a shape that has six points positioned on straight lines coupling vertices of the honeycomb shape to a center of the opening and a point positioned at a greater distance from the center of the opening than at least one of the six points.

8. The flexible hollow structure according to claim 1,
wherein when the opening of each hollow cell section is seen in a plan-view perspective, the opening has an approximately star shape.

9. An adhesive sheet comprising:
the flexible hollow structure according to claim 1; and
an adhesive provided in contact with the opening of the flexible hollow structure.

10. The adhesive sheet according to claim 9,
wherein the adhesive is also provided inside each hollow cell section of the flexible hollow structure near the opening.

11. A functional adhesive sheet comprising:
the adhesive sheet according to claim 9; and
a functional material provided in each hollow cell section of the flexible hollow structure of the adhesive sheet.

12. The functional adhesive sheet according to claim 11,
wherein the functional material comprises a drug.

13. The functional adhesive sheet according to claim 11,
wherein the functional material is provided in some of the plurality of hollow cell sections of the flexible hollow structure, and
wherein the adhesive is provided in contact with the opening of each hollow cell section that is not provided with the functional material.
